(19) United States
(12) Patent Application Publication
(10) Pub. No.: US 2002/0114802 A1
(43) Pub. Date: Aug. 22, 2002

(54) Oral immunoglobulin treatment for inflammatory bowel disease

(76) Inventors: Bo Arthur Einar Tjellstrom, Norrkoping (SE); Lars Christian Stenhammar, Linkoping (SE); Karl-Eric Ivan Magnusson, Linkoping (SE); Tommy Eugen Sundqvist, Linkoping (SE)

Correspondence Address: SCULLY, SCOTT, MURPHY & PRESSER, 400 Garden City Plaza, Garden City, NY, 11530, US

(21) Appl. No.: 09/925,671

(22) Filed: Aug. 9, 2001

Related U.S. Application Data
(63) Continuation-in-part of application No. 09/247,396, filed on Feb. 10, 1999, now abandoned.
(60) Provisional application No. 60/074,193, filed on Feb. 10, 1998.

(51) Int. Cl. A61K 39/395

(52) U.S. Cl. 424/141.1

(57) ABSTRACT

The present invention provides a method of treating inflammatory bowel disease (IBD) in a patient in need thereof which comprises orally administering to the patient an effective amount of a pooled human polyclonal immunoglobulin preparation. The method allows treatment of mucosal inflammation from the luminal side of the gastrointestinal mucosa. Human immunoglobulin preparations suitable for use in the methods of the present invention may be made by any of the well-known methods used for preparing intravenous and intramuscular (parenteral) immunoglobulin preparations. Suitable immunoglobulin preparations may also be obtained commercially. The human immunoglobulin preparation may comprise any of the known immunoglobulin classes including IgA, IgG, IgM, IgE, and IgD. Preferably, the human immunoglobulin preparation comprises at least one of immunoglobulin G (IgG), immunoglobulin A (IgA) or a mixture of immunoglobulin G (IgG) and immunoglobulin A (IgA).

This application is a continuation-in-part of U.S. application Ser. No. 09/247,396, which claims priority from U.S. Provisional Application No. 60/074,193 filed on Feb. 10, 1998.

### BACKGROUND OF THE INVENTION

Inflammatory bowel disease (IBD) refers to serious, chronic disorders of the intestinal tract and specifically includes ulcerative colitis (UC) and Crohn's disease. IBD is often confused with irritable bowel syndrome (IBS). IBS refers to a wide spectrum of digestive problems ranging from common discomfort after eating, to diarrhea, constipation, alternating diarrhea and constipation, or any of these with abdominal pain. Lin Chang et al., 1994 IM-Internal Medicine (USA)15/2 (pp. 27-30, 32-34). In IBS, unlike in IBD, there is no inflammatory component. In fact, examination of the tissues of the intestine and colon either by x-ray, scope, or biopsy reveals no abnormalities. The symptoms of IBS appear to be related to disturbances in gastrointestinal motility. While IBS is a serious condition, it is not an autoimmune disease as is IBD. Approximately 500,000 Americans are afflicted with IBD, and there are about 40,000 new cases of IBD diagnosed each year in the U.S. Although IBD encompasses both UC and Crohn's disease, the two diseases differ in their pathology. In UC, the inner lining of the large intestine (colon or bowel) and rectum becomes inflamed. Inflammation usually begins in the rectum and lower (sigmoid) intestine and spreads upward to the entire colon. UC rarely affects the small intestine except for the ileum. Patients suffering from Crohn's disease exhibit a different pattern of inflammation and ulceration. Crohn's disease often produces a patchy type of inflammation that is deeper into the intestinal wall than the superficial inflammation of UC. Furthermore, Crohn's disease can involve any portion of the digestive tract, including the small and large intestine, the stomach, and even the esophagus. The deeper inflammation of Crohn's disease leads to complications such as large hemorrhoids, rectal abscesses, or fistula that are not seen in UC.

The cause of IBD is unknown. Lofberg, R. (1997) J. Int. Med. 241:1-4. Many theories exist, but none have been proven. One leading theory suggests that some infectious agent, possibly a virus or bacterium, interacts with the immune system to trigger an inflammatory reaction in the intestinal wall. More recently, evidence has been mounting to support the role of genes in the development of the disease. Such evidence includes the increased chance of developing IBD in relatives of IBD patients, the clustering of the disease within families, the association of IBD with other genetic syndromes, and ethnic variations in disease frequencies. Since multiple genes are most likely involved with the development of IBD, it is difficult both to accurately predict which family members will develop IBD, and to identify the genes involved. Thus far, four chromosomal regions have been implicated as contributing to IBD susceptibility. Of these, a region of chromosome 16 has been confirmed.

Recent advances in understanding the role of the immune system in IBD have come from the establishment of strains of mice that develop intestinal inflammation because of the loss of specific genes controlling the immune response. The development of these animal models indicates that aberrant functioning of T cells could be responsible for the development of IBD. The chronic inflammation which is found in IBD may be the result of continuing development and activation of T-helper 1 (TH1) cells in the gut-associated immune system. This conclusion is supported by the observation of enhanced TH1 cell activity in patients with Crohn's disease.

Sufferers of Crohn's disease exhibit inflammation of the intestinal mucosa and submucosa which likely affects the normally tight junctions between adjacent enterocytes. When viewed under the light microscope, however, the enterocytes appear intact (MacDonald, T. T., et al. 1994 Balliere's Clin. Gastroenterol. 8:1-34). An impaired mucosal barrier has been shown in adults and children with Crohn's disease, and interestingly, even in the relatives of Crohn's patients who have not yet developed the disease (Hollander, D., et al. 1986 Ann. Intern. Med. 105:883-885).

Treatment of IBD typically begins with steroids and 5-aminosalicylic acid (5-ASA) drugs. The majority of patients (about 70%) respond well to steroids and 5-ASA drugs. Both of these treatments however, have problems associated with their use that often make long-term use impractical. Furthermore, approximately 30% of IBD patients do not respond well to either therapy, and require other treatment programs such as immunosuppressant agents and surgery. Because of the very individual nature of IBD, no one treatment program is best, and treatment must be tailored to each individual patient. Hanauer, S. B. (1996) New Eng. J. Med. 334(13):841-848. Furthermore, because IBD is not a curable disease (except for surgery in the case of UC), patients are generally required to be maintained on long-term treatment therapies in order to maintain remission. Even surgery is not a cure in Crohn's disease, since the disease tends to recur even after intestinal resection, almost always at the site of surgery.

Steroids remain the first line treatment for IBD, especially during acute phases of the disease. Prednisone and prednisolone are the two most common steroid treatments for IBD. Other steroids in use include methylprednisolone (Medrol®) and budesonide. While showing early results, budesonide is not effective in maintaining remission for more than six months. Because of the severe side effects associated with steroids (hypertension, pancreatitis, osteoporosis, endocrine irregularities, glaucoma), they often are not viewed as an effective long-term maintenance therapy, although many IBD patients do take steroids for long periods of time (long-term being greater than 3-4 months). Sutherland, L. R. (1997) Can. J. Gastroenterol. 11(3):261-264. Steroids continue to be in widespread use, however, because of their effectiveness in acute situations, their success in treating the majority of IBD patients, and their relatively low cost. When properly administered, steroids are an affective treatment, whose side effects can generally be managed and treated with other interventions.

Sulfasalazine is an effective maintenance medication in the treatment of mild to moderate ulcerative colitis, while oral measalamine (Asacol®, Pentasa®, Rowasa®), and 5-ASA derivatives (Dipentum®) are effective in preventing relapses in patients with Crohn's disease. Sulfasalzine and 5-ASA agents are in use because they have a much safer side effect profile than steroids. Side effects associated with 5-ASA agents are not common, but do include pancreatitis and hair loss. The major drawback to use of 5-ASA agents is their high cost. In addition, sulfa drugs and 5-ASA agents provide little relief for the 30% severe IBD patients.

Steroids, sulfasalazine, and 5-ASA drugs can be considered the “classic” treatments for IBD, and for about 70% of IBD patients these treatments, when properly administered, are often the best treatments. However, 30% of the IBD patient population exists that does not respond well to either treatment course. These are the severe, high-risk, patients for whom other treatment programs such as surgery and immunosuppressant agents are required. Sandborn, W. J. (1996) Am. J Gastroenterol. 91(3):423-432.

The use of immunosuppressant therapy in the treatment of IBD is increasing in order both to overcome the long-term side effects of steroid therapy, and to treat the 30% of patients who do not respond to either steroids or 5-ASA agents. Of the immunosuppressant drugs currently is use, Imuran and Purinethol are the most widely prescribed for IBD patients, and are effective in maintaining remission in both UC and Crohn's patients. Methotrexate has also been used, but has been shown to maintain remission poorly in Crohn's disease, and not at all in UC. Cyclosporin has been effective in treating UC, but only in high doses which makes it unsafe to use. Cyclosporin has not been effective in Crohn's disease.

The side effects associated with the long-term use of immunosuppressants can be severe. Adverse side effects include neoplasia (Imuran®), leukopenia and thrombocytopenia, serious infection, bone marrow toxicity (Purinethol®), and hepatotoxicity (Purinethol®). Another major drawback to the use of immunosuppressants is the lag time before they become effective (often six to twelve months) in IBD patients.

Surgery is performed only in the most severe cases of UC and Crohn's disease. For UC patients in which the entire colon is involved, the risk of colon cancer is as high as 32 times the normal rate. Surgery for UC is usually indicated because of massive bleeding, chronic debilitating illness, perforation of the colon, or cancer risk. About 20% of UC patients eventually require either colectomy or proctocolectomy. Surgery for Crohn's disease offers only temporary relief because of the tendency for the disease to recur.

Treatment of IBD by intravenous administration of immunoglobulins has been investigated (Levine D. S., et al., 1992 Am. J Gastroenterol. 87:91 -100; Wolf A., et al., 1988 Mschr. Kinderheilk 136:101-103; Knoflach P., et al., 1990 Ann. Intern. Med. 1 12:385-386; Schmidt, C., 1990 Klinikarzt 19:552-558; Canva-Delcambre, V. 1996 Aliment. Pharmacol. Ther. 10:721-727) with inconsistent results.

Despite the different therapies currently available, there is a need for more effective methods of treating IBD with fewer accompanying side effects. The present invention provides effective methods for treating both UC and Crohn's disease by oral administration of a human immunoglobulin preparation.

### SUMMARY OF THE INVENTION

The present invention provides a method of treating inflammatory bowel disease (IBD) in a patient in need thereof which comprises orally administering to the patient an effective amount of a pooled human polyclonal immunoglobulin preparation. The method allows treatment of mucosal inflammation from the luminal side of the gastrointestinal mucosa. Human immunoglobulin preparations suitable for use in the methods of the present invention may be made by any of the well-known methods used for preparing intravenous and intramuscular (parenteral) immunoglobulin preparations. Suitable immunoglobulin preparations may also be obtained commercially. The human immunoglobulin preparation may comprise any of the known immunoglobulin classes including IgA, IgG, IgM, IgE, and IgD. Preferably, the human immunoglobulin preparation comprises at least one of immunoglobulin G (IgG), immunoglobulin A (IgA) or a mixture of immunoglobulin G (IgG) and immunoglobulin A (IgA). The immunoglobulin preparation is preferably dispersed in a pharmaceutically acceptable carrier and orally administered in a dose of from about 0.5 to 1.5 grams at least once a day. In accordance with the present invention, oral administration of a human immunoglobulin preparation may be done alone or in combination with other treatment regimes.

### BRIEF DESCRIPTIONS OF THE DRAWINGS

FIG. 1 graphically depicts mucosal barrier characteristics before (closed symbol) and after (open symbol) oral immunoglobulin treatment in the patient of Example 1, as assessed with the 6-h urinary recovery of different-sized polyethylene glycols (mol. weights 282-1030 Da).

FIG. 2 graphically depicts mucosal barrier characteristics before (closed symbol) and after (open symbol) oral immunoglobulin treatment in the patient of Example 2, as assessed with the 6-h urinary recovery of different-sized polyethylene glycols (mol. weights 282-1030 Da).

### DETAILED DESCRIPTION OF THE INVENTION

In accordance with the present invention, it has been surprisingly discovered that mucosal inflammation associated with IBD can be effectively treated by oral administration of a pooled human polyclonal immunoglobulin (IG) preparation. In one embodiment mucosal inflammation associated with IBD can be effectively treated from the luminal side of the mucosa by oral administration of a pooled human polyclonal immunoglobulin (IG) preparation. The present invention therefore, is useful for preventing, inhibiting, and/or ameliorating inflamed and impaired portions of the gastrointestinal tract in patients suffering from IBD.

More specifically, in accordance with the present invention, a patient suffering from IBD is treated by orally administering a therapeutically effective amount of a pooled human immunoglobulin preparation for a time and under conditions sufficient to prevent, inhibit, and/or ameliorate mucosal inflammation and impairment in that portion of the gastrointestinal tract affected by the disease.

As used herein a “pooled human polyclonal immunoglobulin preparation” refers to an immunoglobulin composition containing polyclonal antibodies obtained from the plasma of thousands of human donors. The polyclonal antibodies of the present invention are non-antigen specific and may include IgG, IgA, IgM, etc. or fragments thereof. A preferred polyclonal fraction contains IgG for treating immune-mediated diseases including ulcerative colitis, for example. A preferred immunoglobulin composition contains at least about 30% to about 85% IgG polyclonal antibodies, about 5% to about 30% IgA and about 1% to about 25% IgM and trace amounts of other components such as, for example, clotting factors II, VII, IX, X and alpha and beta globulins. Another preferred immunoglobulin composition contains about 95% to about 99% IgG polyclonal antibodies, at least 0.01% to about 2% IgM and trace amounts of salt. Still another preferred immunoglobulin composition contains at least about 25% IgG polyclonal antibodies, at least about 5% to about 30% IgA and about 1% to about 25% IgM, together with trace amounts of clotting factors II, VII, IX, alpha and beta globins and lipids.

As used herein, “treating” and “treatment” refer to administering to a patient a therapeutically effective amount of a pooled human polyclonal immunoglobulin preparation so that mucosal inflammation is prevented, inhibited and/or ameliorated. The term “subject” as used herein, is taken to mean any mammalian patient to which a pooled human polyclonal immunoglobulin preparation is orally administered according to the methods described herein. In a preferred embodiment, the methods of the present invention are administered to a human subject.

The immunoglobulins to be administered orally in accordance with the methods of the present invention may be prepared from human blood using the same procedures that are used in preparing immunoglobulins for intramuscular (parenteral) or intravenous administration. Methods for making intramuscular immunoglobulin (IMIG) preparations and intravenous immunoglobulin (IVIG) preparations are well known in the art. Normally, immunoglobulins for use in IMIG and IMIV preparations are pooled from human volunteers and may comprise varying amounts of the five classes of immunoglobulins; IgA, IgG, IgM, IgE and IgD. Preferably, an immunoglobulin preparation suitable for use in the methods of the present invention is made up of predominantly IgG or IgA immunoglobulins, or a mixture of IgG and IgA immunoglobulins.

Thus, immunoglobulins for oral administration for use in practicing the methods of the present invention may be prepared by Cohn fractionation (Cohn et al., 1946, J Am. Chem. Soc. 68:459-475; Oncley et al., 1949, J. Am. Chem. Soc., 71:541-550),or the method of Kistler and Nitschmann (1962 Vox Sang 7:414-424), ultracentrifugation (Barundem et al., 1962 Vox Sang. 7:157-174), pH adjustments (Koblet et al., 1976 Vox Sang.,31:141-151), fractionation (Schneider et al., 1976 Vox Sang.31: 141-151), enzymatic modification (Fahey et al., 1963 J Exper. Med.,118:845-868; Kneapler et al., 1977 Vox Sang.,32:159-164), structural modification (Barundem et al., 1975 Mong. Allergy 9:39-60), chemical modification (Stephan, Vox Sang. 1975 28:422-437, Masuko et al., 1977 Vox Sang. 32:175-181), reduction and alkylation (U.S. Pat. No. 3,903,262 to Pappenhagen et al.), polyelectrolyte affinity adsorption, large scale electrophoresis, ion exchange adsorption, and polyethylene glycol fractionation. Any method which fractionates immunoglobulins from a human source may be used to obtain immunoglobulins suitable for use in practicing the methods of the present invention. In one embodiment the immunoglobulin preparation is produced by cold alcohol (e.g., ethanol) fractionation from the plasma of about 1000 to about 3000 human volunteers according to the Cohn's method 6 (Cohn, et al., 1946 J Am. Chem. Soc., 66:459-475, incorporated herein by reference).

Additional preparative steps may be used in order to ensure the safety of an immunoglobulin preparation for use in the methods of the present invention. Standards for the preparation of IVIG were proposed in 1989 in a World Health Organization (WHO) bulletin and updated in 1989 to increase the safety of prepared immunoglobulins and other blood products. The immunoglobulin preparations for use in the methods of the present invention may be rendered safe for oral administration using the same methods used for rendering safe immunoglobulin preparations for intravenous administration (IVIG). Such methods are well known in the art and include enzymatic hydrolysis, chemical modification via reduction and alkylation, sulfonation, treatment with—propiolactone, treatment at low pH, purification by ion exchange chromatography, treatment with solvent/detergent and pasteurization. Descriptions of these methods can be found e.g. in Romer J., et al., 1982 Vox Sang. 42:62-73; Romer J., et al., 1990 Vox Sang. 42:74-80; and Rutter G. H. 1994 J. Neurosurg. Psychiat. 57 (Suppl.):2-5.—propiolactone in particular, has proven very effective in eliminating a number of enveloped and nonenveloped viruses including hepatitis C and human immunodeficiency virus (HIV)(Dichtelmuller, H. 1993 Biologicals 21:259-268; Stephan, W., 1975 J Med. Virol. 26:227-232). The disclosure of this article and of all other articles and patents cited in this application are incorporated herein as if fully set forth.

Immunoglobulins for use in practicing the methods of the present invention may also be obtained through commercial sources. Such sources include but are not limited to: BayRho-D® Full Dose (Bayer Biological), BahRho-D® Mini-Dose (Bayer Biological), Gamimune N®, 5% (Bayer Biological), Gamimune N®, 5% Solvent/Detergent Treated (Bayer Biological), Gamimune N®, 10% (Bayer Biological), Gammagard S/D® (Baxter Healthcare), MICRhoGAM® (Ortho Diagnostic), RhoGAM® (Ortho Diagnostic), Sandoglobulin I.V.® (Novartis), Polygam S/D® (American Red Cross), Venoglobulin-S® 5% Solution Solvent Detergent Treated (Alpha Therapeutic),Venoglobulin-S® 10% Solution Solvent Detergent Treated (Alpha Therapeutic), VZIG® (American Red Cross), IgAbulin® (Immuno AG, Vienna, Austria) and Intraglobin-F® (Biotest Pharma GmbH, Frankfurt, Germany). The commercial source of immunoglobulin preparation for use in the methods of the present invention is not critical as studies have shown that different commercial products for intravenous use in different applications performed equivalently. Schiff, R. I., et al. 1977 J. Clin. Immun. 17(1):21-28; Haque, K. N., et al., 1995 Clin. Exp. Immunol 101:328-333.

The orally administrable pharmaceutical compositions for use in practicing the methods of the present invention comprise a pooled human polyclonal immunoglobulin preparation in a therapeutically effective amount in a pharmaceutically acceptable carrier with or without an inert diluent. The carrier should be assimilable and edible and includes liquid, semi-solid, e.g. pastes, or solid carriers. The use of such carriers enables formulation in hard or soft shell gelatin capsules, tablets, pills, or an elixir, suspension, syrup or the like. Enteric coated tablets, capsules or pills are especially helpful in preventing possible denaturation of immunoglobulin in the stomach or upper bowel. Except insofar as any conventional media, agent, diluent or carrier is incompatible with the immunoglobulin preparations of the present invention, its use in an orally administrable immunoglobulin for use in practicing the methods of the present invention is contemplated.

Requirements for a carrier, diluent, media or agent in the immunoglobulin preparation for use in the methods of the present invention are that it not harm the recipient, that it not be detrimental to the immunoglobulin and that the immunoglobulin be stable therein. Examples of carriers or diluents include fats, oils, water, lipids, liposomes, resins, binders, fillers and the like, or combinations thereof. The immunoglobulin may be combined with the carrier by solution, suspension, emulsification, admixture, encapsulation, absorption, adsorption and the like. The carrier should protect the integrity of the immunoglobulin molecule thereby maintaining its therapeutic effectiveness. The term “therapeutic effectiveness” refers to the immunoglobulin preparation being effective for the prevention, inhibition, and/or amelioration of disease symptoms associated with IBD such as mucosal inflammation as exhibited by impaired mucosal barrier characteristics.

A stabilizing agent may also be incorporated into the pharmaceutical compositions for use in the methods of the present invention in order to protect the immunoglobulin from loss of therapeutic activity through, e.g., denaturation. Examples of stabilizers for use in an orally administrable immunoglobulin preparation include buffers, antagonists to the secretion of stomach acids, amino acids such as glycine and lysine, carbohydrates such as dextrose, mannose, galactose, fructose, lactose, sucrose, maltose, sorbitol, mannitol, etc., proteolytic enzyme inhibitors, and so forth.

The precise therapeutically effective amount of immunoglobulin preparation to be administered can be determined by a physician with consideration of individual differences in age, weight, and disease symptoms. It can generally be stated that in practicing the methods of the present invention, an immunoglobulin preparation should be administered in a dose of from about 500 mg to about 5 grams at least once a day. In a preferred embodiment, a dose range of from about 0.5 grams to 1.5 grams is administered from one to three times a day. The time needed to complete a course of the treatment can be determined by a physician and may range from as little as one day to more than one week. A preferred course of treatment is from 2 to 6 weeks. In a more preferred embodiment, a course of treatment lasts for four weeks. A course of treatment may be repeated as often as necessary, as determined by a physician, in order to alleviate recurring symptoms.

The immunoglobulin preparations useful for practicing the methods of the present invention may comprise about 1-100% immunoglobulin. In a more preferred embodiment, IgG and IgA are the predominant immunoglobulins in the preparation. The compositions useful for practicing the methods of the present invention may also contain other immunoglobulins such as IgM, IgD, and IgE. For example, IgAbulin®, an appropriate commercial immunoglobulin preparation (Immuno AG, Vienna, Austria), contains 90 mg of immunoglobulin (of which 60 mg are IgA), per milliliter. Another commercial source of IG, appropriate for use in the methods of the present invention is Sandoglobuling®, (Novartis) which contains 96% IgG with traces of IgA and IgM.

The methods of the present invention may be performed on IBD patients in conjunction with conventional treatments for ulcerative colitis or Crohn's disease. Thus, IBD patients can undergo the methods of the present invention during the course of other treatment procedures, i.e., administration of steroid, 5-ASA, and other drugs. The methods of the present invention may also be administered during the course of enteral nutrition treatments, i.e., either before, after, or simultaneously with such treatments.

In another aspect of the invention, oral administration of immunoglobulins according to the methods of the present invention may be performed on IBD patients after other, conventional procedures such as drug therapy and/or surgery have been suspended or completed.

Patients treated according to the methods of the present invention exhibit reduced mucosal inflammation measurable by improved mucosal barrier characteristics. To monitor such improved mucosal barrier characteristics, various well-known assays can be performed. For example, intestinal permeability may be measured immediately before and after immunoglobulin treatment, using a 6-hour urinary recovery of a mixture of polyethylene glycol (PEG) 500 and 1000 (molecular weight range 2282-1250 Da). This procedure is well known and discussed in Stenhammar L., et al.1989 J. Pediatr. Gastroenterol. Nutr. 9:281-289; and Falth-Magnusson, K., et al., 1984 Clin. Allergy 14:277-286. Using this assay, patients undergoing treatment in accordance with the methods of the present invention exhibit higher levels of large-sized PEGs before treatment and lower levels of large-sized PEGs after treatment. Patients treated according to the methods of the present invention may have an improved condition according to other indicia as well, such as a decreased incidence of blood-stained stools, diarrhea and abdominal cramping.

The invention is further illustrated by the following specific examples which are not intended in any way to limit the scope of the invention.

### EXAMPLE 1

The effect of oral administration of an IG preparation was studied in an 18-year-old girl who had been diagnosed with Crohn's disease engaging the small bowel at 14 years of age,(based on internationally recognized criteria published by Holmquist, et al., 1988 Scand. J Gastroenterol 23:577-84). Prior to the oral administration of immunoglobulin, the patient had received continuous treatment with sulphasalazine and periodical treatment with prednisolone. Since diagnosis she had been hospitaized 12 times because of relapse.

As part of the case study, the patient was treated with 1 g of sulphasalazine daily and 15 mg of prednisolone once daily. The patient had just completed a 4-week treatment with nocturnal enteral whole-protein nutrition without adequate clinical improvement. Table 1 lists the relevant laboratory investigations performed on this patient. Along with the sulphasalazine and prednisolone medication, the patient was orally administered 14 ml of IgAbulin® (Immuno AG, Vienna, Austria) three times daily for 4 weeks. The IgAbulin used in this study had an IG concentration of 90 mg per ml, of which 60 mg were IgA. The patient's condition improved gradually during this period.

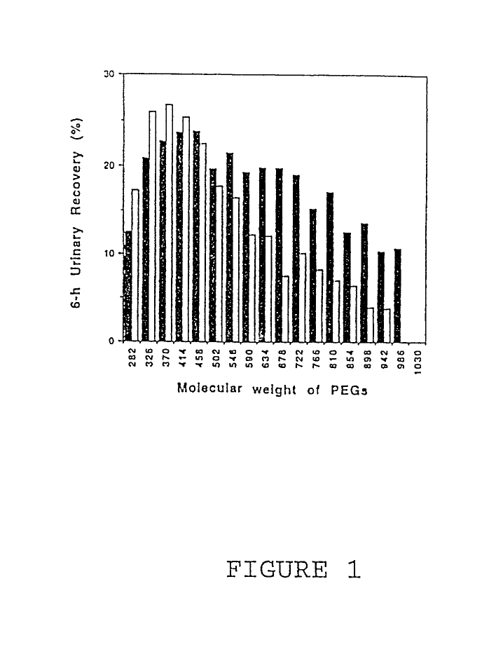

To assess whether the intestinal mucosal barrier characteristics had been affected, a 6 hour urinary recovery of a mixture of polyethylene glycols(PEG) 500 and 1000(molecular weight range 282-1250 Da)(Stenhammar L., et al., 1989; Falth-Magnusson, K., et al., 1984) was performed to probe the intestinal permeability immediately before and after IgAbulin treatment. After treatment, there was less recovery of large-sized PEGs, indicating an improvement of the mucosal barrier (FIG. 1.).

### EXAMPLE 2

The effect of oral administration of an IG preparation was studied in a 19-year-old girl who, at the age of 14 years, had developed inflammatory changes in her buccal mucosa, similar to sarcoidosis. Further investigations revealed Crohn's disease with small bowel and colonic engagement fulfilling the diagnostic criteria formulated by Holmquist et al. 1988. On admission she was treated with sulphasalazine and had a history of blood-stained stools for several months but no diarrhea or abdominal pains. Table 1 lists the relevant laboratory investigations performed on this patient. Maintaining the sulphasalazine medication, she was also treated with 14 ml of IgAbulin® orally three times daily for 4 weeks. Her stools became gradually less blood-stained and her general condition improved.

TABLE 1

| | Patient of | Patient of | | |
|---|---|---|---|---|
| Laboratory Test | Example 1 | Example 2 | Reference Values | |
| ESR | 33 | 48 | 2-15 | mm |
| CRP | 26 | 67 | <3 | mgl⁻¹ |
| Hemoglobin | 88 | 108 | 120-160 | gl⁻¹ |
| Thrombocytes | 623 | 482 | 180-350 × 10⁹ | l⁻¹ |
| S-Albumin | 38 | 28 | 36-48 | gl⁻¹ |
| S-Orosomucoid | 1.07 | 1.68 | 0.29-0.79 | gl⁻¹ |
| S-Iron | Not Tested | 2 | 14-29 | μmoll⁻¹ |

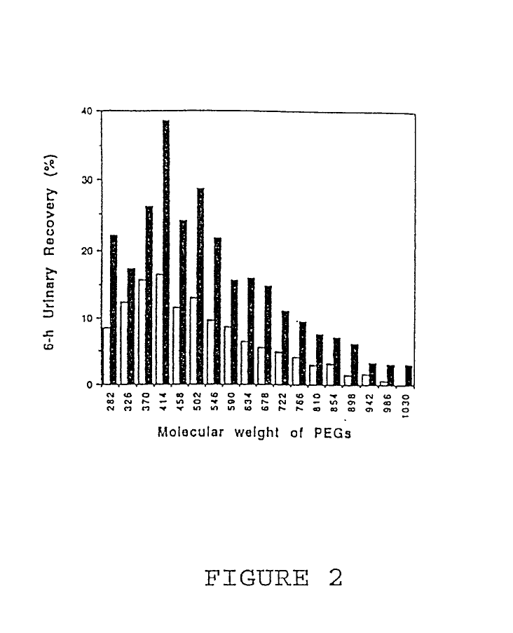

The oral PEG test displayed lower urinary recovery of probes after treatment with immunoglobulin, indicating an improved mucosal barrier as in Example 1 (FIG. 2).

What is claimed is:

1. A method of treating inflammatory bowel disease (IBD) in a patient in need thereof which comprises orally administering to the patient an effective amount of a pooled human polyclonal immunoglobulin preparation.

2. The method according to claim 1 wherein the inflammatory bowel disease is ulcerative colitis (UC).

3. The method according to claim 1 wherein the inflammatory bowel disease is Crohn's disease.

4. A method of treating mucosal inflammation which comprises orally administering to a patient suffering from said mucosal inflammation an effective amount of a pooled human polyclonal immunoglobulin preparation.

5. The method according to any one of claims 1-4 wherein the immunoglobulin is at least one of immunoglobulin G (IgG), immunoglobulin A (IgA) or a mixture of immunoglobulin G (IgG) and immunoglobulin A (IgA).

6. The method according to any one of claims 1-4 wherein the immunoglobulin preparation is dispersed in a pharmaceutically acceptable carrier.

7. The method according to any one of claims 1-4 wherein the amount of immunoglobulin administered to said patient is from about 0.5 to 1.5 grams at least once a day.

8. The method according to any one of claims 1-4 wherein the immunoglobulin is enterically coated.

9. A method of treating inflammatory bowel disease (IBD) in a patient in need thereof which comprises orally administering to the patient a pooled human polyclonal immunoglobulin preparation comprising at least about 25% IgG polyclonal antibodies.

10. A method of treating mucosal inflammation which comprises orally administering to a patient suffering from said mucosal inflammation a pooled human polyclonal immunoglobulin preparation comprising at least about 25% IgG polyclonal antibodies.

11. A method of treating inflammatory bowel disease (IBD) in a patient in need thereof which comprises orally administering to the patient a pooled human polyclonal immunoglobulin preparation comprising at least about 30% to about 85% IgG, about 5% to about 30% IgA and about 1% to about 25% IgM polyclonal antibodies.

12. A method of treating mucosal inflammation which comprises orally administering to a patient suffering from said mucosal inflammation a pooled human polyclonal immunoglobulin preparation comprising at least about 85% IgG, about 5% to about 30% IgA and about 1% to about 25% IgM polyclonal antibodies.
